(19) United States
(12) Patent Application Publication
(10) Pub. No.: US 2025/0172706 A1
(43) Pub. Date: May 29, 2025

(54) METHOD FOR VISUALIZING SCATTERED RADIATION AND MEDICAL SYSTEM

(71) Applicant: Siemens Healthineers AG, Forchheim (DE)

(72) Inventors: Philipp Bernhardt, Forchheim (DE); Niko Köster, Neunkirchen am Brand (DE); Boris Stowasser, Erlangen (DE)

(21) Appl. No.: 18/960,176

(22) Filed: Nov. 26, 2024

(30) Foreign Application Priority Data
Nov. 27, 2023 (DE) 10 2023 211 824.6

(51) Int. Cl. G01T 1/205; A61B 6/00; A61B 6/40; A61B 6/42; A61B 6/46; G01T 1/20

(52) U.S. Cl. CPC G01T 1/205; A61B 6/40; A61B 6/42; A61B 6/461; A61B 6/485; G01T 1/2018

(57) ABSTRACT

Systems and methods for visualizing scattered X-rays is provided for protecting medical staff during an examination with X-rays when an examination object is irradiated with X-rays emitted by an X-ray tube of an X-ray device. The method includes irradiating the examination object with the X-rays emitted by the X-ray tube, thereby producing scattered X-rays, ascertaining signals representing radiation from an initial radiation spectrum, wherein the radiation from an initial radiation spectrum has been produced by scintillation of the scattered X-rays in a gaseous scintillator in the form of nitrogen present in the ambient air, and outputting at least one image ascertained from the signals.

### CROSS REFERENCE TO RELATED APPLICATIONS

This application claims the benefit of DE 10 2023 211 824.6 filed on Nov. 27, 2023, which is hereby incorporated by reference in its entirety.

### FIELD

Embodiments relate to a method for visualizing scattered radiation.

### BACKGROUND

In medical X-ray-based imaging, irradiation of an examination object (for example an organ or part of a patient's body) produces scattered radiation that is distributed throughout the room. To protect those present, for example the attending physician and other medical staff, the scattered radiation should be shielded, but this is not always possible. For this reason, it is at least important to know the exact location and intensity of the scattered radiation in the room in order to avoid exposure as far as possible.

As a rule of thumb, scattered radiation is mainly generated on the side of the examination object, i.e. at the point where the primary radiation enters the examination object (the patient). Furthermore, scattered radiation may also be scattered on all other surfaces or objects and reflected back.

### BRIEF SUMMARY AND DESCRIPTION

The scope of the present disclosure is defined solely by the claims and is not affected to any degree by the statements within this summary. The present embodiments may obviate one or more of the drawbacks or limitations in the related art. Independent of the grammatical term usage, individuals with male, female or other gender identities are included within the term.

Embodiments provide a method that provides for scattered radiation to be visualized as intuitively as possible.

The method for visualizing scattered X-rays when an examination object is irradiated with X-rays emitted by an X-ray tube of an X-ray device includes the following steps: irradiating the examination object with the X-rays emitted by the X-ray tube, thereby producing scattered X-rays, ascertaining signals representing radiation from an initial radiation spectrum, wherein the radiation from an initial radiation spectrum has been produced by scintillation of the scattered X-rays in a gaseous scintillator in the form of nitrogen present in the ambient air, and outputting at least one image ascertained from the signals.

Ambient air is always present and generally contains about 78.08% nitrogen by volume. Embodiments utilizes the scintillation effect of the nitrogen present in the ambient air in combination with the scattered X-rays, as a result of which radiation from an initial radiation spectrum, mainly including ultraviolet radiation, is generated. The distribution of the radiation from the initial radiation spectrum generated in this way (for example including ultraviolet radiation) corresponds to the distribution of the scattered X-rays. Ultraviolet radiation may in turn be visualized and recorded and thus be used to indicate and visualize the scattered X-rays.

Visualizing the scattered X-rays allows members of the medical staff to see and avoid precisely those areas with high radiation exposure. This enables hazards and risks to be reduced or completely eliminated. Measures may also be taken to protect exposed areas by shielding. Visualization may also be used as training material for medical staff. Embodiments help to protect the health of the medical staff during X-ray examinations and interventional procedures under X-ray monitoring.

According an embodiment, the radiation from an initial radiation spectrum is filtered with respect to visible residual light by at least one filter element and the radiation component formed by UV radiation is converted by at least one conversion element into visible light. This enables the visualization of the ultraviolet radiation to be simplified or enhanced. First, the ambient light, which may impair the signals, is filtered out; this may, for example, be accomplished using known filter elements for filtering out visible radiation. The ultraviolet radiation is then converted into visible light, for example using a fluorescent screen, since recording of visible light is straightforward and easy to perform.

Since the visible light generated in this way may be very weak, according to an embodiment, the radiation component converted into visible light is amplified. For example, a residual light amplification element may be used for this purpose. This enables the signal to be enhanced so that the light distribution is clearly visible to a user.

According to a further embodiment, the amplified radiation component converted into visible light is imaged onto a sensor. Further optical elements, such as, for example, mirrors, prisms or filters may be used for this purpose.

The converted visible light is recorded by the sensor and converted into a 2D or 3D image of the radiation distribution. The sensor may, for example, be formed by a known image sensor. The sensor may, for example, have a plurality of sensor elements. The sensor may, for example, be part of a digital camera, a UV camera or digital glasses, for example VR glasses or AR glasses. Image sensors and their mode of operation are known, they are readily available and technologically advanced and are therefore straightforward and easy to use.

According to a further embodiment, the at least one 2D or 3D image ascertained in this way is displayed on a display unit, for example on a monitor, a touchpad or a computer screen. This allows one or more users to see the distribution of the scattered X-rays precisely and identify which areas are exposed to high levels of radiation. In the case of digital glasses (VR glasses or AR glasses), the 2D or 3D image is displayed directly in the glasses, i.e. the display unit is part of the glasses.

The at least one 2D or 3D image is stored for later use or display, for example in a memory unit assigned to the receiving unit or in a database.

According to a further embodiment, a 2D or 3D image of the room (i.e. for example the spatial area around the examination object) is additionally recorded and displayed together with the 2D or 3D image of the radiation distribution. These may be displayed next to one another in at least partial superimposition or as overlapping in order to facilitate assignment. This allows the user to easily recognize the distribution of the scattered X-rays in the room.

Embodiments also include a medical system for performing the method for visualizing scattered X-rays, the medical system including: an X-ray device with an X-ray tube configured to emit X-rays to irradiate an examination object, a receiving unit for ascertaining signals representing radiation from an initial radiation spectrum, wherein the radiation from an initial radiation spectrum has been produced by scintillation of the scattered X-rays in a gaseous scintillator in the form of nitrogen present in the ambient air, and an output unit for outputting at least one image ascertained from the signals. The receiving unit may, for example, be a suitable 2D or 3D camera (for example a UV camera or conventional camera). The receiving unit may also be formed by digital glasses with or without augmented reality functions.

The recording device may include at least one filter unit for filtering out residual light and a conversion unit for converting the radiation from the initial radiation spectrum (especially ultraviolet radiation) into visible light. The conversion unit may, for example, be a fluorescent screen. The recording device may additionally include a residual light amplifier. According to a further embodiment, the receiving unit also has a sensor for recording the signals and converting them into a 2D or 3D image of the radiation distribution, for example an image sensor. The receiving unit and/or the output unit may be configured as a digital camera, a digital UV camera or digital glasses, for example VR or AR glasses.

The medical system also includes a memory unit for storing the image or is connected to a database for storage.

According to a further embodiment, the medical system includes a second receiving unit, for example a camera, for recording a 2D or 3D image of the room.

According to an embodiment, the X-ray device may include an X-ray detector for recording two-dimensional or three-dimensional X-ray images of the examination object.

### BRIEF DESCRIPTION OF THE FIGURES

FIG. 1 depicts a view of an emission spectrum of nitrogen exposed to ionizing radiation.

FIG. 2 depicts a sequence of processes triggered by X-rays according to an embodiment.

FIG. 3 depicts a sequence of steps of a method for visualizing scattered radiation according to an embodiment.

FIG. 4 depicts a further sequence of steps of a method for visualizing scattered radiation according to an embodiment.

FIG. 5 depicts a view of a medical system for performing a method for visualizing scattered radiation according to an embodiment.

FIG. 6 depicts an enlarged view of a receiving unit of a medical system according to FIG. 5 according to an embodiment.

### DETAILED DESCRIPTION

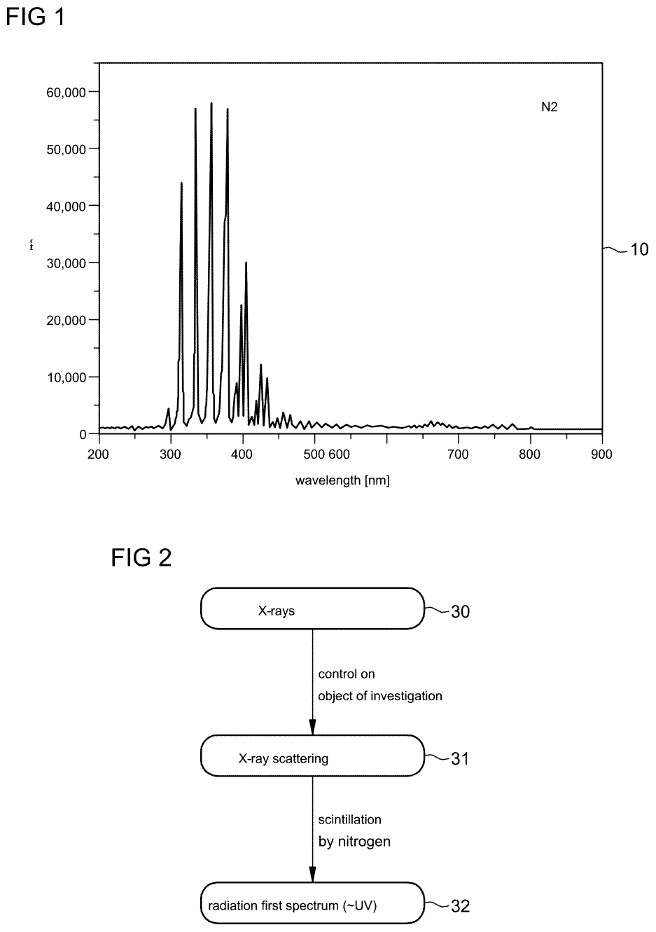

FIG. 1 shows an emission spectrum 10 produced when high-energy radiation interacts with nitrogen. Herein, the peaks produced are largely located in the ultraviolet region of the spectrum, which means that herein characteristically ultraviolet radiation is generated. FIG. 2 shows the sequence of the resulting processes. When X-rays 30 interact with an object, for example the examination object, scattering produces scattered X-rays 31 which are distributed in the room. These are generally unfocussed and their distribution in the room is unknown to the medical staff, which makes it difficult for them to protect themselves from scattered X-rays. If the scattered X-rays 31 interact with nitrogen atoms, the resulting scintillation effect converts them into radiation 32 from an initial radiation spectrum, which mainly includes ultraviolet radiation.

Since ambient air is always present, except in a vacuum, and generally contains about 78.08% nitrogen by volume, the ambient air may serve as a nitrogen source. Embodiments utilizes the scintillation effect of the nitrogen in the ambient air in combination with scattered X-rays and visualizes the corresponding radiation from an initial radiation spectrum which mainly includes ultraviolet radiation. A scintillator re-emits part of the absorbed energy in the form of light in all spatial directions. Nitrogen acts as such a scintillator. Wherever primary and scattered radiation deposits energy in the ambient air, nitrogen emits UV radiation isotropically. Depending upon the intensity of the radiation, more or less UV radiation is released.

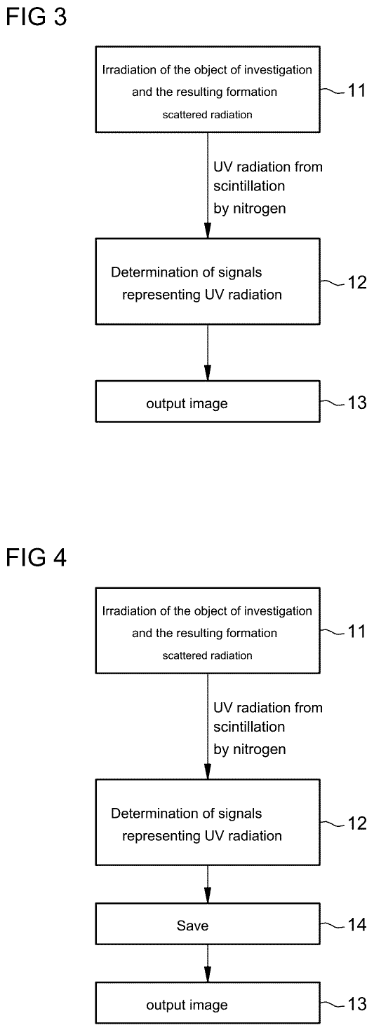

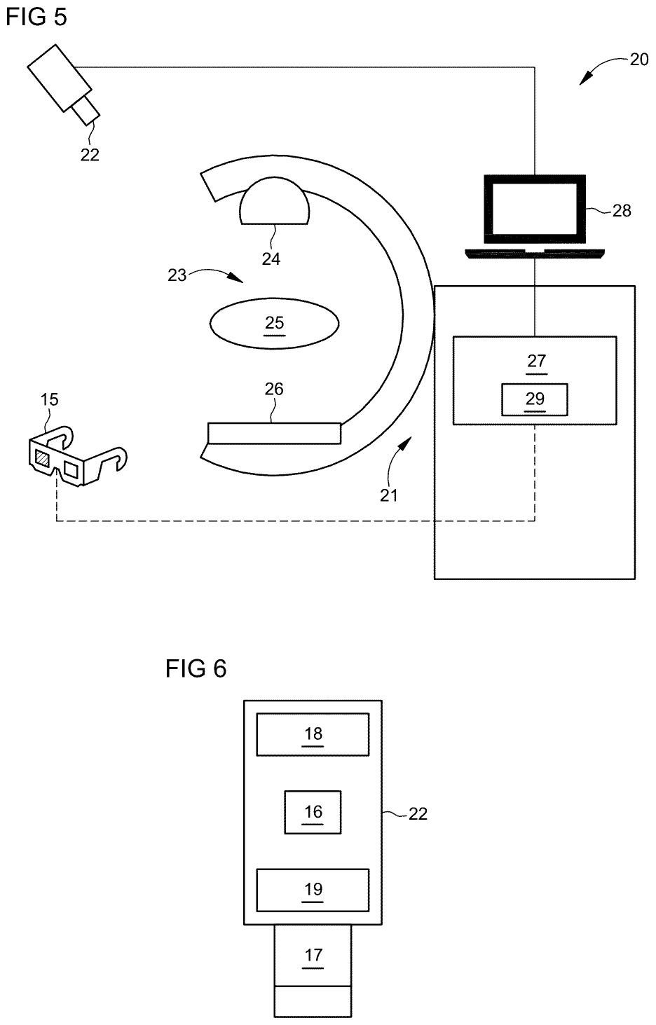

FIG. 3 depicts steps of the method for visualizing scattered X-rays when an examination object 25 is irradiated with X-rays 30 emitted by an X-ray source 24 of an X-ray device 21. FIG. 5 depicts a medical system 20 for performing the method with a UV camera and FIG. 6 shows an enlarged view of the UV camera 22. In a first step 11, the examination object 25 is irradiated with X-rays 30 emitted by the X-ray source 24 of the X-ray device 21. The X-rays 30 may, for example, be triggered when one or more X-ray images are recorded. The irradiation of the examination object 25 produces scattered X-rays 31. The scattered X-rays 31 ionize nitrogen molecules in the ambient air and these are excited to emit radiation 32 from an initial radiation spectrum. This radiation 32 from the initial radiation spectrum may, for example, resemble the emission spectrum shown in FIG. 1 and mainly has ultraviolet radiation.

Signals representing the radiation 32 from the initial radiation spectrum are then ascertained from the radiation 32 from the initial radiation spectrum in a second step 12. One (or more) digital UV camera(s) 22 or digital UV glasses 15 may be used for this purpose, for example. The following detailed steps may be performed using such tools: first, filtering takes place, for example by one or more filter elements of a residual light filter 17 so that the visible light is filtered out. This serves, among other things, to filter out ambient light present in the room that has not been produced by scintillation. It is also possible for the residual light filter 17 used to be a dichroic mirror which is configured to reflect visible light and allow UV radiation to pass through.

The radiation component formed by UV radiation is then converted into visible light by at least one conversion element. Such a conversion element may, for example, be a known fluorescent screen 19 or another fluorescent element such as those usually used to convert ultraviolet radiation into visible light.

Since the visible light generated in this way may be very weak, it is amplified, for example using a residual light amplification element 16. This may enhance the signal strength, making it easier for users (for example medical staff) to recognize or assign the light distribution. The light (signal) amplified in this way is then imaged onto a sensor 18. Further optical elements, such as, for example, mirrors, prisms or filters may be used for this purpose. The sensor 18 may, for example, be formed by a known image sensor, wherein the sensor 18 may, for example, have a plurality of sensor elements. The sensor 18, the residual light filter 17, the fluorescent screen 19 and the residual light amplification element 16 may, for example, be part of the digital UV camera 22 or digital UV glasses 15 (for example digital amplification glasses which are configured to detect UV radiation with or without an AR function). Image sensors and their mode of operation are known, they are readily available and technologically advanced and are therefore straightforward and easy to use.

The amplified light (signal) is recorded by the sensor 18 and converted into a 2D or 3D image which may be used to visualize the distribution of the scattered X-rays 31.

In a third step 13, at least one such 2D or 3D image is output. The image may be output directly, for example via the digital UV glasses 15, in that a user wearing the digital UV glasses 15 may directly see the radiation distribution through the lenses acting as a display. Users may wear such digital glasses 15 for teaching purposes or also permanently, for example during an examination or a procedure on a patient under X-ray monitoring. In the case of prolonged use, users may always see the distribution of the scattered X-rays 31 and constantly rethink and optimize their behavior, for example by changing position in the room or improving the shielding. If the display lens is transparent, the image may be displayed superimposed in the correct position in relation to the environment as augmented reality. When using a UV camera 22 or another camera, an image may, for example, also be output on a display unit, for example a monitor 28 or a touch screen or smart device, for example after being stored in a memory unit 29 or a database (fourth step 14 in FIG. 4). The display unit may be part of the medical system 20 which also includes the X-ray device 21 for the examination.

It is also possible to use a stereo camera or also two or more UV cameras 22 at different positions in the room in order to generate a three-dimensional image.

The receiving unit used may also be a modified DSLR camera (digital single-lens reflex camera) in which a standard sensor cover that filters UV light has been removed. This enables the corresponding sensor to be sensitive to at least 365 nm. A DSLR camera is known. In addition, special forensic lenses that are UV-permeable may be used for this DSLR camera. A filter element that is permeable to ultraviolet radiation may be arranged in front of the special lens. This combination may be used to ascertain and output images from the radiation from the initial radiation spectrum generated by nitrogen scintillation.

In addition to the image of the distribution of the scattered X-rays, it is also possible to record a two-dimensional or three-dimensional image of the spatial environment of the examination object 25. This may be performed with one or more conventional cameras which operate in the visible range. The image may then be displayed together with the image of the distribution of the scattered X-rays, for example side by side or partially or completely superimposed. When the images are superimposed in the correct position, the spatial relationship of the radiation distribution to the examination object and the medical system is easy to recognize. With digital glasses, superimposition may also be part of augmented reality or virtual reality.

The medical system 20 is actuated by a system controller 27. This may control the triggering of the X-rays 30. In addition, image processing, display and further processes may also be actuated by the system controller. In addition to the X-ray source 24, the X-ray device 21 also has an X-ray detector 26 which generates an X-ray image from the X-rays 30 passing through the examination object 25.

To protect medical staff during an examination with X-rays, a method is provided for visualizing scattered X-rays when an examination object is irradiated with X-rays emitted by an X-ray tube of an X-ray device, including the following steps: irradiating the examination object with the X-rays emitted by the X-ray tube, thereby producing scattered X-rays, ascertaining signals representing radiation from an initial radiation spectrum, wherein the radiation from an initial radiation spectrum has been produced by scintillation of the scattered X-rays in a gaseous scintillator in the form of nitrogen present in the ambient air, and outputting at least one image ascertained from the signals.

It is to be understood that the elements and features recited in the appended claims may be combined in different ways to produce new claims that likewise fall within the scope of the present disclosure. Thus, whereas the dependent claims appended below depend from only a single independent or dependent claim, it is to be understood that the dependent claims may, alternatively, be made to depend in the alternative from any preceding or following claim, whether independent or dependent, and that such new combinations are to be understood as forming a part of the present specification.

While the present disclosure has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A method for visualizing scattered X-rays when an examination object is irradiated with X-rays emitted by an X-ray tube of an X-ray device, the method comprising:
irradiating the examination object with the X-rays emitted by the X-ray tube, thereby producing scattered X-rays;
ascertaining signals representing radiation from an initial radiation spectrum, wherein the radiation from an initial radiation spectrum has been produced by scintillation of the scattered X-rays in a gaseous scintillator in a form of nitrogen present in ambient air; and
outputting at least one image ascertained from the signals.

2. The method of claim 1, wherein the radiation from an initial radiation spectrum is filtered with respect to visible residual light by at least one filter element and a radiation component formed by UV radiation is converted into visible light by at least one conversion element.

3. The method of claim 2, wherein the radiation component converted into visible light is amplified.

4. The method of claim 3, wherein the visible light is amplified by a residual light amplification element.

5. The method of claim 3, wherein the amplified radiation component converted into visible light is imaged onto a sensor.

6. The method of claim 5, wherein the converted visible light is recorded by the sensor and converted into a 2D image or a 3D image of a radiation distribution.

7. The method of claim 6, wherein the 2D or the 3D image is displayed on a display unit, stored in a memory unit, or displayed on the display unit and stored in the memory unit.

8. The method of claim 6, further comprising:
recording and displaying a 2D image or 3D image of a room together with the 2D or the 3D image of the radiation distribution.

9. A medical system for visualizing scattered X-rays, the medical system comprising:
an X-ray device including an X-ray tube configured to emit X-rays to irradiate an examination object,
a receiving unit configured for ascertaining signals representing radiation from an initial radiation spectrum, wherein the radiation from an initial radiation spectrum is produced by scintillation of the scattered X-rays in a gaseous scintillator in a form of nitrogen present in ambient air, and
an output unit for outputting at least one image ascertained from the signals.

10. The medical system of claim 9, wherein the receiving unit has at least one filter element for filtering out residual light and a conversion element for converting UV radiation into visible light.

11. The medical system of claim 10, wherein the conversion element comprises a fluorescent screen.

12. The medical system of claim 9, wherein the receiving unit includes a residual light amplification unit.

13. The medical system of claim 9, wherein the receiving unit includes a sensor for recording light and converting the light into a 2D image or a 3D image of a radiation distribution.

14. The medical system of claim 9, wherein the receiving unit and/or the output unit are configured as a camera, an ultraviolet (UV) camera, virtual reality (VR) glasses, or augmented reality (AR) glasses.

15. The medical system of claim 9, wherein the X-ray device includes an X-ray detector for recording X-ray images of the examination object.

16. The medical system of claim 9 further comprising:
a memory unit configured for storing the at least one image.

17. The medical system of claim 9, further comprising:
a second receiving unit configured for recording a 2D image or 3D image of a room.
